(19) United States
(12) Patent Application Publication
(10) Pub. No.: US 2009/0098191 A1
(43) Pub. Date: Apr. 16, 2009

(54) Use of bases to stabilize transdermal formulations

(76) Inventors: Christopher G. Anderson, Salt Lake City, UT (US); Sonia J. Heiber, Salt Lake City, UT (US); Blaine Travis Messenger, Salt Lake City, UT (US); Peter Hart, Lehi, UT (US)

Correspondence Address: Hedman and Costigan PC, 1185 Avenue of the Americas, Ste 2003, New York, NY, 10036, US

(21) Appl. No.: 11/975,126

(22) Filed: Oct. 16, 2007

(51) Int. Cl. A61K 31/56; A61K 9/70; A61P 43/00

(52) U.S. Cl. 424/443; 514/182

(57) ABSTRACT

Stabilized transdermal acid sensitive drug formulations and their associated methods of production and use are described. The formulations containing acid sensitive drugs can also an acrylic adhesive, and a pharmaceutically acceptable base. The formulations can further contain other adhesives, permeation enhancers, and other stabilizing compounds such as free radical inhibitors.

### FIELD OF THE INVENTION

The present invention relates to formulations and methods for their manufacture in the preparation of stabilized transdermal drug delivery formulations. Accordingly, this invention involves the fields of chemistry, pharmaceutical sciences, medicine and other health sciences.

### BACKGROUND OF THE INVENTION

Various drugs when included in transdermal formulations are susceptible to degradation by various pathways. Drugs such as norelgestromin (NG) contain an oxime moiety that is susceptible to degradation via a hydrolytic pathway (subject to general acid catalysis). Oxidation of C-6 and C-10 within the steroid nucleus of norelgestromin also occurs and is subject to acid catalysis. Many other 3-keto-4-ene steroids used as drugs are also susceptible to oxidation at these positions (C-10 oxidation is prevalent with only C-19 desmethylsteroids) as well as at C-9 in the case of A ring aromatic steroids, for example, ethinylestradiol (EE).

In the case of the above molecules, as well as many other drugs which are susceptible to oxidation, acid hydrolysis or general acid catalyzed degradation promoted by the presence of catalytic amounts of acid in a formulation, can destabilize the drugs and shorten shelf-life. That is, the rate of degradation of these molecules is substantially increased in the presence of acid or acidic sites or moieties (both protonic and Lewis acids).

### SUMMARY

Accordingly, the present invention provides formulations and methods for stabilizing acid sensitive drugs in transdermal formulations. In one aspect, a stabilized transdermal drug formulation is provided. The stabilized transdermal drug formulation can include an acid sensitive drug, an acrylic adhesive, and a pharmaceutically acceptable base.

In another embodiment, a method of providing effective contraception in a female subject is provided. The method provides for administering an amount of norelgestromin sufficient to achieve AUC values of from 190 ng/ml to 205 ng/ml from a transdermal patch to the subject. The transdermal patch can include a therapeutically effective amount of norelgestromin, an acrylic adhesive, and a pharmaceutically acceptable base.

In another aspect, a method of making a stabilized drug delivery patch may be provided. The method can include forming a transdermal formulation by admixing an amount of an organic base with an acrylic adhesive containing an acid sensitive drug. The transdermal formulation of acrylic adhesive and acid sensitive drug can then be incorporated into a patch device that can act as a source of the acid sensitive drug.

In a further aspect, a method of stabilizing an acid sensitive drug in an acrylic adhesive-containing patch may be provided which includes inhibiting the degradation of an acid sensitive drug by formulating the acrylic adhesive-containing patch to contain a pharmaceutically acceptable base.

### DEFINITIONS OF KEY TERMS

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “an excipient” includes reference to one or more of such excipients.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, the terms “formulation” and “composition” are used interchangeably and refer to a mixture of two or more compounds, elements, or molecules. In some aspects the terms “formulation” and “composition may be used to refer to a mixture of one or more active agents with a carrier or other excipients. The terms “drug,” “pharmaceutical,” “active agent,” and “bioactive agent” are also used interchangeably to refer to a pharmacologically active substance or composition. These terms of art are well-known in the pharmaceutical and medicinal arts.

As used herein, “transdermal” refers to the route of administration taken by a drug that is applied to and absorbed through an area of skin. In some aspects, the skin may be substantially unbroken. Thus the terms “transdermal formulation” and “transdermal composition” can be used interchangeably, and refer to formulations or compositions that are applied to a surface of the skin and transdermally absorbed. Examples of transdermal formulations include but are not limited to, ointments, creams, gels, transdermal patches, sprays, lotions, mousses, aerosols, nasal sprays, buccal and sublingual tablets and tapes, vaginal rings, and pastes. The term “transdermal administration” thus refers to the transdermal application of a formulation or composition. Transdermal administration can be accomplished by applying, pasting, rolling, attaching, pouring, pressing, rubbing, etc., of a transdermal preparation or formulation onto a skin or mucosal surface. These and additional methods of administration are well-known in the art.

The terms “transdermal delivery system,” “transdermal patches” or simply “patches” refer to a matrix or liquid reservoir type of transdermal delivery device which is used to transdermally deliver defined doses of a substance, over a specific application period.

As used herein, “enhancement,” “penetration enhancement,” or “permeation enhancement,” refer to an increase in the permeability of the skin to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which is incorporated by reference herein.

The term “admixed” means that the drug and/or enhancer can be dissolved, dispersed, or suspended in the carrier. In some cases, the drug may be uniformly admixed in the carrier.

As used herein, “acid sensitive” when used to describe active agents or drugs refers to a drugs propensity to degrade in the presence of acids or acidic groups or moieties. For example, an “acid sensitive drug” can include sex steroids, such as norelgestromin.

As used herein, “hybrid adhesives” refers to pressure sensitive adhesives that are comprised of a copolymer or mixture of copolymers that contain two or more monomers within the chains that are from different general structural types. For example an adhesive prepared by copolymerization of an acrylate monomer (first general structural type) with a monomer such as isobutylene or rubber type monomer (second general structural type) would produce a hybrid acrylate/rubber type adhesive. Any combination of dissimilar monomeric combinations to form a copolymer that acts as a pressure sensitive adhesive is included in the definition. Other examples of potential combinations include acrylate/polyurethane, polyurethane/rubber, acrylate/styrene, styrene/rubber, etc. An example of a commercially available hybrid adhesive is DURO-TAK® 87-502A manufactured by National Starch. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

### DETAILED DESCRIPTION

The present invention involves the use of pharmaceutically acceptable bases in order to form stabilized transdermal drug formulations, particularly when acrylic adhesives are included in the formulation. Since acrylic adhesives are polymers containing a wide variety of ester and amide functionalities of monomeric acrylic acid, as well as some metallic cross-linkers like titanium and aluminum, they are not normally considered to be acidic. However it has been discovered that the presence of a very small number of free carboxyl groups or metals in these polymers can catalyze degradation of certain drugs. Specifically, it has been discovered that when acrylic adhesives are incorporated into transdermal drug delivery systems, such as transdermal patches, the acrylic adhesives can promote degradation of acid sensitive drugs. Further, it has also been discovered that through the addition of a catalytic or larger amount of a pharmaceutically acceptable base to acrylic adhesive-containing transdermal formulations, such acid sensitive drugs are stabilized and the degradation thereof is meaningfully slowed or stopped. Without being limited by theory, it is believed that such stabilization results from the neutralization of the acidic sites within the formulation.

When acrylic adhesives are used in the transdermal formulations of the present invention they can comprise from about 0.05 wt % to 90 wt % of the formulation. In one embodiment, the acrylic adhesive can comprise from about 0.75 wt % to 5 wt % of the formulation. In another embodiment, the acrylic adhesive can comprise about 1 wt % of the formulation.

Acrylic adhesives are well known in the transdermal drug delivery arts. Generally, any acrylic adhesive can be used in the formulations of the present invention. The acrylic adhesive can be an acrylic copolymer adhesive. Non-limiting examples of acrylics adhesives which can be used include DURO-TAK®acrylic adhesives made by National Starch, including DURO-TAK® 87-2516, DURO-TAK ELITE® 87-900A, and the like. In one aspect of the present invention, the acrylic adhesive can be non-curing. In another embodiment, the acrylic adhesive contains a titanium cross-linker.

In addition to the stabilization of formulations containing acrylic adhesives, the present invention may also be used to stabilize formulations containing other acidic components which might otherwise cause degradation of an acid sensitive drug. The addition of a pharmaceutically acceptable base to a transdermal formulation will generally stabilize acid sensitive drugs without substantially affecting the performance of the transdermal device. Simply stated, the invention involves the use of bases to stabilize transdermal formulations that contain acid sensitive drugs. However, the invention is not limited by the mechanism of degradation, whether it is due to neutralization of acidic sites or some other unknown mechanism that is inhibited by the addition of base.

In one aspect, a stabilized transdermal drug formulation is provided. The stabilized transdermal drug formulation can include an acid sensitive drug, an acrylic adhesive, and a pharmaceutically acceptable base.

In another aspect, a method of making a stabilized drug delivery patch may be provided. The method can include forming a transdermal formulation by admixing an amount of an organic base with an acrylic adhesive containing an acid sensitive drug. The transdermal formulation of acrylic adhesive and acid sensitive drug can then be incorporated into a patch device which can act as a source of said acid sensitive drug.

In a further aspect, a method of stabilizing an acid sensitive drug in an acrylic adhesive-containing patch may be provided which includes inhibiting the degradation of the acid sensitive drug by formulating the acrylic adhesive-containing patch to contain a pharmaceutically acceptable base.

The present invention can be used to stabilize formulations containing a wide range of acid sensitive drugs. The amount of acid sensitive drug in the stabilized transdermal drug formulations of the present invention can vary depending on the type of acid sensitive drug and the targeted or desired affect. For example, in one embodiment, the acid sensitive drug can comprise from about 0.1 wt % to about 10 wt % of the total formulation. In another embodiment, the acid sensitive drug can comprises from 0.25 wt % to about 3 wt % of the formulation.

In one embodiment, the acid sensitive drug can be a sex steroid. Non-limiting examples of sex steroids include norelgestromin, ethinyl estradiol, progesterone, ethisterone (17-α-ethinyltestosterone), medroxyprogesterone, hydroxyprogesterone, norethindrone (17-α-ethinyl-19-nortestosterone), norethindrone acetate (17-α-ethinyl-19-nortestosterone acetate), dydrogesterone (9-β-10-α-pregna-4,6-diene-3,20-dione), dimethisterone (6-α-methyl-17-α-[1-propynyl]-testosterone), chlormadinone acetate (6-chloro-6-dehydro-17-α-acetoxyprogesterone), norgestrel (13-β-ethyl-17-α-ethinyl-17-β-hydroxygon-4-en-3-one), testosterone, methyltestosterone, fluoxymesterone, methandrostenolone, nandrolone, norethandrolone, hydroxycortisone, cortisone, desoxycorticosterone, fludrocortisone, betamethasone, dexamethasone, prednisolone, prednisone, methylprednisolone, paramethasone, triamcinolone, flumethasone, fluocinolone, fluocinonide, fluprednisolone, halcinonide, flurandrenolide, meprednisone, medrysone, norethynodrel, esters thereof, and combinations thereof.

In one embodiment, the sex steroid can be norelgestromin. When present in the stabilized transdermal formulation, the norelgestromin can comprise from about 0.5 wt % to about 5 wt % of the formulation. In one aspect, the norelgestromin can comprises from about 1.5 wt % to about 2.5 wt % of the formulation.

As discussed above, stabilization of the acid sensitive drugs in the transdermal drug formulations can be accomplished through the incorporation of a pharmaceutically acceptable base to the formulation. Examples of pharmaceutically acceptable bases which can be used include, but are not limited to, triethanolamine, tromethamine, diisopropanolamine, stearamidoethyldiethylamine, ethanolamine, diethanolamine, physiologically compatible organic bases, inorganic bases such as lithium, sodium potassium, calcium and magnesium hydroxides, bicarbonates, carbonates as well as salts of week acids such as sodium acetate and sodium benzoate and other salts of physiologically compatible organic acids, and mixtures thereof. In one embodiment, the pharmaceutically acceptable base can comprise from about 0.005 wt % to about 0.5 wt % of the formulation. In another embodiment, the pharmaceutically acceptable base can comprise from about 0.01 wt % to about 0.25 wt % of the formulation. In one embodiment, the pharmaceutically acceptable base can be an organic base. In one aspect, the pharmaceutically, acceptable base can be triethanolamine. When triethanolamine is included in the formulation it can comprise from about 0.01 wt % to about 0.1 wt % of the total formulation. In another aspect, the pharmaceutically, acceptable base can be tromethamine.

In addition to acrylic adhesives, the transdermal formulations of the present invention can also include other known transdermal adhesives. Generally, any type of transdermal adhesive known in the transdermal arts can be used including but no limited to polyisobutylene adhesives, polystyrene adhesives, hybrid adhesives, polyurethane adhesives. In one embodiment, the transdermal adhesive includes a polyisobutylene adhesive.

The transdermal formulations can also include permeation enhancers. Non-limiting examples of permeation enhancers which can be used include lauryl lactate, isopropyl myristate, methyl laurate, oleyl alcohol, glycerol monooleate, glycerol dioleate, glycerol trioleate, glycerol monostearate, glycerol monolaurate, propylene glycol monolaurate, water, diols, mono-alcohols, DMSO, dimethylformamide, N,N-dimethylacetamide, 2-pyrrolidone, azones, mixtures thereof and mixtures thereof. In one embodiment, the permeation enhancer is lauryl lactate. When present in the formulation, lauryl lactate can comprise 1 wt % to 5 wt % of the formulation.

The transdermal formulations can include other stability enhancing agents known in the art so long as they do not interfere with the stabilization effects of the pharmaceutically acceptable base. In one embodiment, a free radical inhibitor can be included in the transdermal formulation. An example of a free radical inhibitor which can be used is butylated hydroxytoluene (BHT).

When the formulations of the present invention are incorporated into a transdermal patch, the transdermal patch can be capable of providing effective contraception in a female subject. Specifically, the patches can administer an amount of norelgestromin sufficient to achieve AUC values of from 185 ng/ml to 205 ng/ml from the transdermal patch. In another embodiment, the patch can provide AUC values of from 190 ng/ml to 200 ng/ml to the subject.

The embodiments and descriptions described above can be applied equally to the formulations of the present invention as well as associated methods of stabilizing acid sensitive drugs, and producing stability enhanced transdermal patches.

### EXAMPLES

The following examples of formulations of norelgestromin and ethinyl estradiol are provided to promote a more clear understanding of certain embodiments of the present invention, and are in no way meant as a limitation thereon.

### Example 1

### Preparation of a_norelgestromin Casting Solution

Norelgestromin, ethinyl estradiol, trolamine, and lauryl lactate were dissolved in a mixture of ethyl acetate and isopropanol in a high-speed mixer container. To the solution was added PVP-CLM and the suspension stirred a minimum of thirty minutes. National Starch DURO-TAK® 87-900A adhesive and DURO-TAK® 87-607A adhesive were added with additional mixing of one hour. The casting solution was transferred to a wide mouth jar for laminating. The casting solution must be rolled to remix prior to use.

### Example 2

### Preparation of_norelgestromin Laminates

A casting solution as prepared in Example 1 was cast onto a release liner with a 20 mil casting knife and dried in a forced air oven at elevated temperature. Two laminates were laminated together with non-woven fabric between the layers and backing film attached. The bilayer laminate was then punched into transdermal systems and sealed in pouches.

### Example 3

### Stability Testing of Norelgestromin Containing Formulations with and without Trolamine

Two norelgestromin formulations, Formulations 1 & 2, were prepared in accordance to Examples 1 and 2, except that Formulation 2 included 0.2 wt % of trolamine as a pharmaceutically acceptable base, while Formulation 1 contained no trolamine. Stability data were collected by HPLC and is shown in Table 1.

TABLE 1

| | Related Substance | Unk 2 | Unk 3 | Unk 4 | Unk 5 | Unk 7 | Unk 9 | Norgestrel | Norgestimate | SUM |
|---|---|---|---|---|---|---|---|---|---|---|
| | Formulation 1 | | | | | | | | | |
| 1% 900A, 0.25% | RRT | 0.39 | 0.41 | 0.42 | 0.44 | 0.46 | 0.47 | 0.96 | 1.39 | |
| EE, 2.0% NG | t = 0 | NSL | ND | 0.08 | ND | ND | 0.058 | 0.128 | ND | 0.266 |
| | 60 C./2 wks | NSL | 0.116 | 0.086 | NSL | 0.095 | ND | 0.151 | ND | 0.461 |
| | Formulation 2 | | | | | | | | | |
| 1% 900A, 0.25% | RRT | 0.39 | 0.41 | 0.42 | 0.44 | 0.44 | 0.47 | 0.96 | 1.39 | |
| EE, 2.0% NG. | t = 0 | NSL | NSL | 0.065 | ND | ND | 0.053 | 0.121 | ND | 0.244 |
| 0.2% TROL | 60 C./2 wks | NSL | NSL | NSL | 0.056 | NSL | ND | 0.158 | ND | 0.218 |

Table 1 shows that the sum of degradation products in the 87-900A formulation containing trolamine at t=0 is less than the formulation containing no trolamine (sum=0.244 vs. 0.266, respectively). At t=2 weeks @ 60° C., the difference is much more significant (sum=0.218 vs. 0.461, respectively).

### Example 4

### Stability Comparisons of Norelgestromin Formulations at 0, 0.05 and 0.2 wt % Trolamine using DURO-TAK® 87-900A and DURO-TAK® 87-607A Adhesives

Norelgestromin formulations, Formulations 1, 2, & 3 were prepared in accordance to Examples 1 and 2, except that Formulation 1 contained no trolamine, Formulation 2, contained 0.05 wt % trolamine, and Formulation 3 contained 0.2 wt % trolamine. Stability data was collected by HPLC and is shown in Table 2.

TABLE 2

| | t = 0 | 60 C./2 wks |
|---|---|---|
| | Total | Total |
| FORMULATION | Degradants | Degradants |
| Formulation 1 | 0.13 | 0.24 |
| 1% 87-900A IN 87-607A, 2% NG, 0.15% | | |
| EE | | |
| Formulation 2 1% | 0.17 | 0.19 |
| 87-900A IN 87-607A, 2% NG, 0.15% EE, | | |
| 0.05% Trolamine | | |
| Formulation 3 1% | 0.11 | 0.11 |
| 87-900A IN 87-607A, 2% NG, 0.15% EE, | | |
| 0.2% Trolamine | | |

NG = norelgestromin;
EE = ethinyl estradiol

Table 2 shows that as the level of trolamine increases from 0% to 0.05 to 0.2% in an 87-900A/87-607A formulation the total degradation products decrease at t=2 weeks, 60° C. It is noteworthy that at a level of 0.2% trolamine in the formulation no difference in total degradation products is seen between t=0 and t=2 weeks, 60° C. samples. The level of 0.05% trolamine may be close to the minimum amount of base necessary to neutralize acidic sites in the 87-900A adhesive.

### Example 5

### Preparation of Norelgestromin/Ethinyl Estradiol Transdermal Systems using DURO-TAK® 87-2516

Norelgestromin/ethinyl estradiol transdermal systems incorporating DURO-TAK® 87-2516 adhesive were formulated with different levels of the chemical stabilizers trolamine (TROL), tromethamine (TRMTH) and butylated hydroxytoluene (BHT) in an adhesive mix in accordance with Examples 1 and 2 to determine if the bases could offer protection against oxidation as does BHT (see Example 6). The formulations were evaluated for impurities and degradation products before and after two weeks of accelerated aging (60° C. storage). In addition BHT systems at t=0 and fourteen day aged systems (60° C.) containing TROL were assayed for the API's and recoveries determined based on coating weights to determine if assay values could be improved by the addition of stabilizers. Each of the test systems set forth in each of the following Examples was hand made with the exception of Lot O5Z201, which was machine made. All hand made test systems were stressed at 60° C. for 2 weeks prior to testing. The systems containing BHT, TROL, and TRMTH were also tested prior to stressing. The prepared dry matrix compositions of salient ingredients are listed below in Table 3. In addition to the 87-2516 acrylic adhesive each formulation also contained lauryl lactate, Crospovidone NF, and polyisobutylene/polybutene (PIB/PB) adhesive (DURO-TAK® 87-607A).

TABLE 3
Test Systems: Prepared Compositions of Key Ingredients

| | w/w | w/w | w/w % | w/w % (chemical |
|---|---|---|---|---|
| Lot | % NG | % EE | (adhesive) | stabilizer) |
| 1322-2108-130- | 1.50 | 0.25 | 3.01 (87-2516) | 0.10 (BHT) |
| 1A | | | | |
| 1322-2108-130- | 1.50 | 0.25 | 3.01 (87-2516) | 0.50 (BHT) |
| 1B | | | | |
| 1322-2108-130- | 1.50 | 0.25 | 3.00 (87-2516) | 1.96 (BHT) |
| 4C | | | | |
| 1322-1995-188-1 | 1.49 | 0.25 | 3.16 (87-2516) | 0.056 (TROL) |
| 1322-1995-190-1 | 1.40 | 0.24 | 2.91 (87-2516) | 0.177 (TROL) |
| 1322-1995-191-1 | 1.50 | 0.25 | 3.04 (87-2516) | 0.507 (TROL) |
| 1322-2099-058-4 | 1.50 | 0.25 | 3.00 (87-2516) | 0.05 (TRMTH) |
| 1322-2099-058-5 | 1.50 | 0.25 | 3.00 (87-2516) | 0.10 (TRMTH) |
| 1322-2099-058-6 | 1.50 | 0.25 | 3.00 (87-2516) | 0.50 (TRMTH) |

### Example 6

### Stability Results for Norelgestromin Formulations Stabilized with BHT

Norelgestromin formulations were prepared similarly to Example 5 using varying amounts of BHT. The stability of the formulations was evaluated after 14 days at T=60° C. Results of the testing are given in Table 4.

TABLE 4

| | | | | 3% | | |
|---|---|---|---|---|---|---|
| | | | | 2516 | 3% 2516 | 3% 2516 |
| Related | | 3% 2516 | Ortho | 0.1% | 0.5% | 2.0% |
| Substance | RRT | No BHT | Evra⁴ | BHT | BHT | BHT |
| Unknown 1 | 0.34 | NSL | ND | ND | ND | ND |
| Unknown 2 | 0.39 | NSL | 0.080 | NSL | NSL | NSL |
| Unknown 3 | 0.41 | 0.153 | NSL | 0.102 | 0.072 | 0.066 |
| Unknown 4 | 0.42 | 0.067 | 0.070 | NSL | NSL | NSL |
| Unknown 5 | 0.44 | 0.190 | 0.100 | 0.102 | 0.066 | NSL |
| Unknown 6 | 0.45 | ND | NSL | 0.060 | NSL | NSL |
| Unknown 7 | 0.46 | 0.097 | NSL | 0.054 | NSL | NSL |
| Unknown 8 | 0.47 | 0.157 | ND | ND | ND | ND |
| Unknown 9 | 0.47 | NSL | NSL | NSL | NSL | NSL |
| Unknown 10 | 0.49 | NSL | 0.090 | NSL | NSL | NSL |
| Unknown 11 | 0.69 | NSL | ND | 0.072 | 0.114 | 0.066 |
| Unknown 12 | 0.87 | NSL | NSL | NSL | ND | ND |
| Norgestrel | 0.96 | 0.583 | 0.120 | 0.450 | 0.312 | 0.240 |
| Unknown 13 | 1.26 | NSL | 0.080 | ND | ND | ND |
| Unknown 14 | 1.27 | ND | ND | ND | ND | ND |
| Norgestimate 2 | 1.39 | NSL | 0.240 | NSL | NSL | ND |
| Unknown 15 | 1.84 | 0.080 | NSL | NSL | NSL | NSL |
| Total at t = 14 | | 1.327 | 0.780 | 0.840 | 0.564 | 0.372 |
| Days | | | | | | |
| Total at t = 0 | | 0.445 | 0.640 | 0.272 | 0.207 | 0.108 |
| Days | | | | | | |

1. Values for individual impurities and degradation products are shown for T = 14 days only.
2. “NSL” (no significant level) means related substance not present at greater than 0.050%.
3. “ND” means no related substance detected.
⁴Ortho Evra had been stored at ambient temperature for 64 weeks at t = 0

It is clear from Table 4 that BHT has a stabilizing affect at all levels on the 2516 formulation with the highest level formulation being the most stable. Totals of degradation products at t=14 days @ 60° C. are lowest for the formulation containing 2% BHT and highest for the formulation containing no BHT. At t=0 days the same trend is obvious. In comparison to Ortho Evra, the other tested formulations containing 0.5% and 2% BHT gave comparable or lower levels of degradation products than Ortho Evra product. The Table shows that the systems containing BHT are more stable and offer the possibility of competing favorably with the stability of Ortho Evra product, particularly at the highest level of BHT. It is noteworthy that high levels of BHT, such as 2 wt %, can cause additional skin irritation.

### Example 7

### Stability Results for Norelgestromin Formulations Stabilized with Trolamine or Tromethamine

Norelgestromin formulations were prepared similarly to Example 5 using varying amounts of trolamine (TROL) or tromethamine (TRMTH). The stability of the formulations was tested after 14 days at T=60° C. The norelgestromin-related impurities/degradation products analysis for NG/EE TDS containing varying levels of the organic bases TRMTH and TROL compared to a base free 2516 formulation and Ortho Evra are given in Table 5, below.

TABLE 5

| | | | | 3% 2516 | 3% 2516 | 3% 2516 | 3% 2516 | 3% 2516 | 3% 2516 |
|---|---|---|---|---|---|---|---|---|---|
| Related | | 3% 2516 | Ortho | 0.056% | 0.177% | 0.507% | 0.05% | 0.10% | 0.5% |
| Substance | RRT | No Base | Evra⁴ | TROL | TROL | TROL | TRMTH | TRMTH | TRMTH |
| Unknown 1 | 0.34 | NSL | ND | ND | ND | NSL | NSL | NSL | ND |
| Unknown 2 | 0.39 | NSL | 0.080 | 0.077 | 0.090 | 0.100 | NSL | NSL | NSL |
| Unknown 3 | 0.41 | 0.153 | NSL | NSL | NSL | 0.083 | 0.230 | 0.160 | 0.083 |
| Unknown 4 | 0.42 | 0.067 | 0.070 | 0.097 | 0.063 | NSL | 0.123 | 0.093 | NSL |
| Unknown 5 | 0.44 | 0.190 | 0.100 | 0.057 | NSL | NSL | 0.173 | 0.107 | 0.067 |
| Unknown 6 | 0.45 | ND | NSL | NSL | NSL | NSL | ND | ND | ND |
| Unknown 7 | 0.46 | 0.097 | NSL | ND | ND | ND | 0.150 | 0.107 | NSL |
| Unknown 8 | 0.47 | 0.157 | ND | NSL | NSL | NSL | NSL | NSL | NSL |
| Unknown 9 | 0.47 | NSL | NSL | NSL | ND | NSL | 0.150 | 0.097 | 0.067 |
| Unknown 10 | 0.49 | NSL | 0.090 | NSL | NSL | NSL | 0.050 | NSL | NSL |
| Unknown 11 | 0.69 | NSL | ND | ND | ND | ND | ND | ND | NSL |
| Unknown 12 | 0.87 | NSL | NSL | ND | ND | NSL | NSL | NSL | NSL |
| Norgestrel | 0.96 | 0.583 | 0.120 | 0.300 | 0.203 | 0.207 | 0.737 | 0.470 | 0.183 |
| Unknown 13 | 1.26 | NSL | 0.080 | NSL | NSL | ND | ND | ND | ND |
| Unknown 14 | 1.27 | ND | ND | ND | ND | ND | ND | ND | ND |
| Norgestimate 2 | 1.39 | NSL | 0.240 | NSL | ND | ND | ND | NSL | ND |
| Unknown 15 | 1.84 | 0.080 | NSL | NSL | NSL | NSL | ND | NSL | NSL |
| Total at t = 14 | | 1.327 | 0.780 | 0.530 | 0.357 | 0.390 | 1.614 | 1.033 | 0.400 |
| Days | | | | | | | | | |
| Total at t = 0 | | 0.445 | 0.640 | 0.277 | 0.140 | 0.273 | 0.370 | 0.330 | 0.330 |
| Days | | | | | | | | | |

1. Values for individual impurities and degradation products are shown for T = 14 days only.
2. “NSL” (no significant level) means related substance not present at greater than 0.050%.
3. “ND” means no related substance detected.
⁴Ortho Evra had been stored at ambient temperature for 64 weeks at t = 0

First, comparing the TRMTH results at the three different levels to the base free 2516 formulation for total related substances at t=14 days, it would appear that in the case of the 0.05% TRMTH formulation more degradation has occurred than in the control (base free 2516). Little difference is seen in degradation level between the three formulations at t=0 days. The 0.1% TRMTH formulation shows decreased degradation at t=14 days, although not much compared to the control, while the 0.5% TRMTH formulation shows a significant improvement in stability compared to control. Since TRMTH has limited solubility in the casting solution and adhesive mix, a possible explanation for the ineffectiveness at the lower levels of TRMTH is that there is simply not enough dissolved base present in the formulation to inhibit degradation, since the bulk of the material is present in suspension rather than solution. At a level of 0.5% TRMTH enough is present to inhibit degradation, whether in solution or suspension. An explanation for why degradation is more significant at the 0.05% TRMTH level compared to control is speculative, but as Table 6 indicates the norgestrel peak in this formulation showed a significant increase compared to control, while the norgestrel peak in the other two formulations showed a decrease. This could indicate that the sample contained water and without enough TRMTH to neutralize acidic groups, the NG would be more labile towards hydrolysis to form norgestrel. Other hydrolytic products could also increase.

Inspection of the TROL results in Table 5 shows that at levels of between ˜0.05% and ˜0.5% TROL degradation of NG is substantially inhibited with the least degradation seen at a intermediate level of 0.177%. It would appear that at both of these levels TROL effectively inhibits degradation of NG. Since the lower level is effective, use in the formulation at a higher level would not be prudent due to the possibility of added skin irritation. The improved effectiveness of TROL compared to TRMTH at all three levels is the result of increased solubility of TROL in the casting solution and adhesive mix compared to TRMTH. Since the TROL is more soluble, it is capable of being uniformly dispersed throughout the solution and more effectively neutralizes acidic sites in solution compared to a suspension of TRMTH.

### Example 8

### Stability of Noregestromin and Ethinyl Estradiol Formulation Stabilized with Triethanolamine and without Triethanolamine

Norelgestromin/ethinyl estradiol transdermal systems containing 0.056 wt %, 0.177 wt %, or 0.507 wt % of triethanolamine were assayed for NG and EE after a period of 23 days at a temperature of 60° C. The results of the testing are set forth in Table 6. It is noted that percent recovery assay is the measured amount of NG or EE divided by the product of the prepared weight fraction of NG or EE in each formulation and the measured coating weight times 100. The coating weight of each individual test system was used to calculate percent recovery of that system.

TABLE 6

| Formulation | % Recovery NG | % Recovery EE |
|---|---|---|
| 0.056% TROL, | 97.6 ± 0.2 | 99.5 ± 0.3 |
| 3% 87-2516 | | |
| 0.177% TROL, | 98.5 ± 1.6 | 97.3 ± 1.4 |
| 3% 87-2516 | | |
| 0.507% TROL, | 98.5 ± 0.6 | 100.5 ± 0.8 |
| 3% 86-2516 | | |

In contrast to the formulation containing triethanolamine, similar formulations containing no stabilizer, namely no triethanolamine, were assayed after a period of 30 days at a temperature of 40° C. Results from the test as set forth in Table 7.

TABLE 7

| Formulation | % Recovery NG | % Recovery EE |
|---|---|---|
| O5Z201 | 93.7 ± 1.3 | 94.5 ± 1.5 |
| 3% 87-2516, No | | |
| TROL | | |

Comparing the recoveries in Table 7 for control systems subjected to accelerated aging at 40° C. for 30 days to those in Table 6 for systems aged 23 days at 60° C. containing three different levels of TROL, it is apparent that the presence of the base at all levels increases NG and EE recoveries. If the TROL had no effect, one would expect lower recoveries for the systems aged under the more harsh conditions of 23 days, 60° C.

### Example 9

### Clinical Study to Evaluate the Bioavailability of Norelgestromin (0.150 MMg/Day)/Ethyinyl Estradiol (0.020 mg/Day Transdermal Delivery System in Normal Healthy Female Volunteers

A study was performed to evaluate the bioavailability of norelgestromin delivered from a transdermal patch containing an acrylic adhesive. Sixty healthy adult female volunteers ranging in age from 18 to 35 and having a body mass index (BMI) between 18-30 kg/m² were chosen. Each of the volunteers was subjected to a 28 day washout period against oral contraceptives including estrogen and progestin combined pills, progestin only pills, or the Ortho Evra® patch and a 6 month washout period against injectable contraceptives.

Following a pre-study examination to confirm healthy conditions, each subject was administered a transdermal patch including an acrylic adhesive. The patches were all applied to a skin region of the upper outer arm which was free of cuts, scratches, and other abrasions. Each patch was applied for a period of 168 hours (7 days) and blood samples were taken at selected intervals of 0, 6, 12, 18, 24, 30, 36, 48, 72, 96, 120, 144, 168, 174, 180, 192, 216, and 240 hours. Plasma from each of the samples was harvested according to standard methodology. The norelgestromin and ethinyl estradiol plasma concentrations were measured using validated bioanalytical methods and according to the PPD's Bioanalytical Laboratory's Standard Operating Procedures and FDA Guidelines.

Tables 8 and 9 set for the bioavailability data for norelgestromin and ethinyl estradiol.

TABLE 8
Summary of Bioavailability Studies for Norelgestromin

| | Subjects¹ | Arithmetic Mean (% CV) Pharmacokinetic | | | | | |
|---|---|---|---|---|---|---|---|
| Treatments | No. (M/F) | Parameters¹ | | | | | |
| (Dose, Dosage | Type | Median (Range) for Tₘₐₓ | | | | | |
| Form, Route) | Age: Mean | Cₘₐₓ | Tₘₐₓ | AUC₀₋ₜ | AUC_{0-∞} | t_{1/2} | kₑ |
| [Product ID] | (Range) | (ng/mL) | (hr) | (hr * ng/mL) | (hr * ng/mL) | (hr) | (1/hr) |
| Test: | 44 (0/44) | 1.35 | 48.00 | 193.51 | 198.38 | 26.37 | 0.0279 |
| Norelgestromin/ | Healthy | (24.33) | (30-120) | (23.36) | (23.94) | (26.09) | (24.61) |
| Ethinyl | Volunteers | | | | | | |
| Estradiol | 24.11 yr | | | | | | |
| 0.150/0.020 mg/ | (18-35 yr) | | | | | | |
| day | | | | | | | |
| Transdermal | | | | | | | |
| System | | | | | | | |
| [Code No. | | | | | | | |
| 3121-1; | | | | | | | |
| Control No. | | | | | | | |
| 06Z111] | | | | | | | |

¹Subjects used in final statistical report

TABLE 9
Summary of Bioavailability Studies for Ethinyl Estradiol

| | Subjects¹ | Arithmetic Mean (% CV) | | | | | |
|---|---|---|---|---|---|---|---|
| Treatments | No. (M/F) | Pharmacokinetic Parameters¹ | | | | | |
| (Dose, Dosage | Type | Median (Range) for Tₘₐₓ | | | | | |
| Form, Route) | Age: Mean | Cₘₐₓ | Tₘₐₓ | AUC₀₋ₜ | AUC_{0-∞} | t_{1/2} | kₑ |
| [Product ID] | (Range) | (pg/mL) | (hr) | (hr * pg/mL) | (hr * pg/mL) | (hr) | (1/hr) |
| Test: | 42 (0/42) | 75.23 | 36.00 | 9947.32 | 10029.15 | 14.86 | 0.0486 |
| Norelgestromin/ | Healthy | (27.08) | (18-144) | (22.46) | (22.23) | (20.06) | (21.12) |
| Ethinyl | Volunteers | | | | | | |
| Estradiol | 23.95 yr | | | | | | |
| 0.150/0.020 mg/ | (18-35 yr) | | | | | | |
| day | | | | | | | |
| Transdermal | | | | | | | |
| System | | | | | | | |
| [Code No. | | | | | | | |
| 3121-1; | | | | | | | |
| Control No. | | | | | | | |
| 06Z111] | | | | | | | |

¹Subjects used in final statistical report

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A stabilized transdermal drug formulation, comprising:
an acrylic containing adhesive;
a therapeutically effective amount of norelgestromin;
a therapeutically effective amount of ethinyl estradiol; and
a pharmaceutically acceptable base, said pharmaceutically acceptable base being present in an amount sufficient that at least about 95 wt % of the norelgestromin remains after the formulation is stored at 60° C. for a period of 23 days.

2. The stabilized transdermal drug formulation of claim 1, wherein the norelgestromin comprises from about 0.5 wt % to about 3 wt % of the formulation.

3. The stabilized transdermal drug formulation of claim 1, wherein the norelgestromin comprises from about 1.5 wt % to about 2.5 wt % of the formulation.

4. The stabilized transdermal drug formulation of claim 1, wherein the ethinyl estradiol comprises from about 0.10 wt % to about 0.5 wt % of the formulation.

5. The stabilized transdermal drug formulation of claim 1, wherein the acrylic containing adhesive contains a titanium cross-linker.

6. The stabilized transdermal drug formulation of claim 1, wherein the pharmaceutically acceptable base is selected from the group consisting of triethanolamine, tromethamine, diisopropanolamine, stearamidoethyldiethylamine, inorganic bases such as lithium, sodium potassium, calcium and magnesium hydroxides and carbonates as well as salts of week acids such as sodium acetate and sodium benzoate, and mixtures thereof.

7. The stabilized transdermal drug formulation of claim 1, wherein the pharmaceutically acceptable base comprises from 0.005 wt % to about 0.5 wt % of the formulation.

8. The stabilized transdermal drug formulation of claim 1, wherein the pharmaceutically acceptable base comprises from 0.01 wt % to about 0.25 wt % of the formulation.

9. The stabilized transdermal drug formulation of claim 1, wherein the pharmaceutically acceptable base is an organic base.

10. The stabilized transdermal drug formulation of claim 1, wherein the pharmaceutically acceptable base is triethanolamine

11. The stabilized transdermal drug formulation of claim 9, wherein the triethanolamine comprises from about 0.01 wt % to about 0.1 wt % of the total formulation.

12. The stabilized transdermal drug formulation of claim 1, wherein the pharmaceutically acceptable base is tromethamine.

13. The stabilized transdermal drug formulation of claim 1, wherein the acrylic adhesive comprises from 0.05 wt % to 90 wt % of the formulation.

14. The stabilized transdermal drug formulation of claim 1, wherein the acrylic adhesive comprises from 0.75 wt % to 5 wt % of the formulation.

15. The stabilized transdermal drug formulation of claim 1, wherein the acrylic adhesive comprises about 1% of the formulation.

16. The stabilized transdermal drug formulation of claim 1, wherein the formulation includes a polyisobutylene adhesive.

17. The stabilized transdermal drug formulation of claim 1, wherein the formulation includes a permeation enhancer selected from the group consisting of lauryl lactate, isopropyl myristate, methyl laurate, oleyl alcohol, glycerol monooleate, glycerol dioleate, glycerol trioleate, glycerol monostearate, glycerol monolaurate, propylene glycol monolaurate, water, diols, mono-alcohols, DMSO, dimethylformamide, N,N-dimethylacetamide, 2-pyrrolidone, azones, mixtures thereof and mixtures thereof.

18. The stabilized transdermal drug formulation as in claim 17, wherein the permeation enhancer is lauryl lactate and the lauryl lactate comprises 1 wt % to 5 wt % of the formulation.

19. The stabilized transdermal drug formulation as in claim 1, wherein the formulation includes butylated hydroxytoluene.

20. The stabilized transdermal drug formulation as in claim 1, wherein the pharmaceutically acceptable base is present in an amount sufficient that at least about 97 wt % of the norelgestromin remains after the formulation is stored at 60° C. for a period of 23 days.

21. A stabilized transdermal drug formulation, comprising:
an acid sensitive sex steroid;
an acrylic adhesive; and
a pharmaceutically acceptable base.

22. The stabilized transdermal drug formulation of claim 1, wherein the acid sensitive sex steroid is norelgestromin.

23. A method of providing effective contraception in a female subject, comprising:
administering an amount of norelgestromin sufficient to achieve AUC values of from 185 ng/ml to 205 ng/ml from a transdermal patch, said transdermal patch comprising a therapeutically effective amount of norelgestromin, an acrylic adhesive, and a pharmaceutically acceptable base.

24. A method of making a stabilized drug delivery patch, comprising, comprising:
forming a transdermal formulation by admixing an amount of an organic base to an acrylic adhesive containing an acrylic sensitive drug; and
incorporating said transdermal drug formulation into said a transdermal patch device as a source of said acid sensitive drug.

25. A method of stabilizing an acid sensitive drug in an acrylic adhesive-containing patch, comprising:
inhibiting degradation of the acid sensitive drug by formulating the acrylic adhesive-containing patch to contain a pharmaceutically acceptable base.
